(19) United States
(12) Patent Application Publication
(10) Pub. No.: US 2011/0172438 A1
(43) Pub. Date: Jul. 14, 2011

(54) Enantioselective Cascade Michael-Michael Reactions and Related Catalysts

(76) Inventor: Wei Wang, Albuquerque, NM (US)

(21) Appl. No.: 13/061,911

(22) PCT Filed: Sep. 8, 2009
(86) PCT No.: PCT/US09/05042

§ 371 Date: Mar. 2, 2011

Related U.S. Application Data
(60) Provisional application No. 61/191,486, filed on Sep. 8, 2008.

(51) Int. Cl. C07F 7/10; C07D 207/04

(52) U.S. Cl. 548/406; 548/572

(57) ABSTRACT

The invention provides direct processes and related catalysts for the syntheses of trisubstituted chiral pyrrolidines, piperidines, tetrahydrothiophenes, and thianes by highly enantio- and diastereoselective cascade Michael-Michael reaction of α, β-unsaturated aldehydes with trans-γ-protected amino α, β-unsaturated esters.

### RELATED APPLICATIONS

This application claims priority from U.S. Provisional Patent Application No. 61/191,486, filed Sep. 8, 2008, the complete contents of which are hereby incorporated by reference.

### FEDERALLY SPONSORED RESEARCH

The present invention was made with government support under NSF grant CHE-0704015 and NIH-INBRE grant P20 RR016480. As a result, the United States retains certain rights in this invention.

### FIELD OF THE INVENTION

The invention relates to processes and related catalysts for the syntheses of trisubstituted chiral pyrrolidines, piperidines, tetrahydrothiophenes, and thianes by highly enantio- and diastereoselective cascade Michael-Michael reaction of α, β-unsaturated aldehydes with trans-γ-protected amino α, β-unsaturated esters.

### BACKGROUND OF THE INVENTION

Citations for all references are found after the experimental section. Throughout this application, various publications are referenced. The disclosures of all of these publications and those references cited within those publications in their entireties are hereby incorporated by reference into this application in order to more fully describe the state of the art to which this invention pertains.

Substituted chiral pyrrolidines and piperidines are ubiquitous structural components of numerous naturally occurring alkaloids and biologically active synthetic substances. (1) For example, trisubstituted pyrrolidines are present in natural products neuroexcitatory amino acids (−)-kainic acid and (+)-α-allokainic acid (2), promising new anti-cancer agent ABT-627 (3) and influenza drug A-192558.4. In addition, the “privileged” structures are the important synthetic target in diversity oriented synthesis. (5) Despite their broad applications, synthetic methods for the efficient preparation of chiral pyrrolidines, piperidines, tetrahydrothiophenes, and thianes are highly limited. The state of the art in chiral pyrrolidine and piperidine syntheses prevalently relies on chiral auxiliary controlled asymmetric synthesis and transition-metal-catalyzed asymmetric dipolar addition reactions. (6) In contrast, organocatalyzed asymmetric processes are largely undeveloped. Only three related examples of employing organocatalyzed enantioselective [3+2] cycloaddition strategy were disclosed recently by Vicario, Córdova and Gong respectively. (7) Furthermore, the asymmetric catalytic synthesis of trisubstituted densely functionalized chiral pyrrolidines, piperidines, tetrahydrothiophenes, and thianes remains elusive and the discovery of catalytic asymmetric reactions that yield such a framework is an important challenge.

Accordingly, the need exists for enantio- and diastereoselective processes that efficiently and practically yield highly-functionalized, trisubstituted chiral pyrrolidines, piperidines, tetrahydrothiophenes and thianes.

### SUMMARY OF THE INVENTION

We have discovered one-pot, high-yield processes and related catalysts for the syntheses of highly-functionalized, trisubstituted chiral pyrrolidines, piperidines, tetrahydrothiophenes, and thianes by a highly enantio- and diastereoselective cascade Michael-Michael reaction of α, β-unsaturated aldehydes with trans-γ-protected amino α, β-unsaturated esters.

In one embodiment, the invention provides processes for synthesizing a compound of the formula 3(a) or formula 3(b):

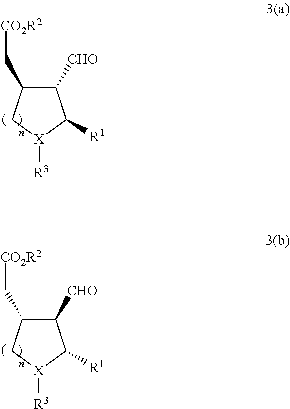

by reacting an α, β-unsaturated aldehyde of formula 1:

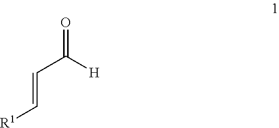

with a trans-γ-protected amino α, β-unsaturated ester of the formula 2 (a) or a trans-γ-mercapto α, β-unsaturated ester of the formula 2 (b):

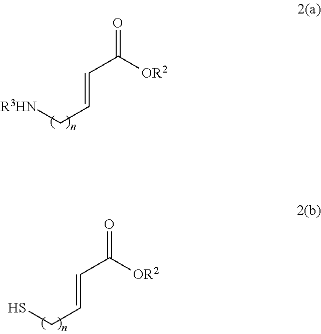

in a reaction medium comprising a weak base, a solvent, and a chiral ether catalyst of the formula 4(a) (to yield compounds of the formula 3(a)) or formula 4(b) (to yield compounds of the formula 3(b)):

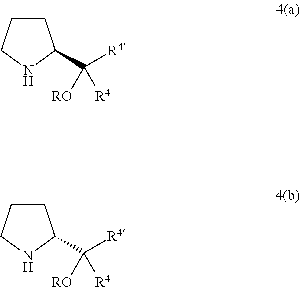

wherein:
n is 1 or 2;

### X is N or S;

R¹ and R² are the same or different and are selected independently from the group consisting of an optionally substituted C₁-C₂₀ alkyl, alkenyl or alkynyl (“hydrocarbyl”) group, an alkoxy group, an optionally substituted aryl group, or an optionally substituted aralkyl group (preferably, R¹ is a phenyl group which is optionally substituted with up to four substituents which are preferably selected from the group consisting of hydroxyl (OH), halogen (preferably F or Cl), CN, NO₂, a C₁-C₆ optionally substituted alkyl group (preferably CH₃, CH₂CH₃ or CF₃), a C₁-C₆ alkoxy group (which group may contain an unsaturated hydrocarbon), or a C₂-C₆ ether group (which group may contain an unsaturated hydrocarbon), and R² is an optionally substituted C₁-C₂₀ alkyl, alkenyl or alkynyl (“hydrocarbyl”) group); where (1) X is N, the α, β-unsaturated aldehyde of formula 1 is reacted with the trans-γ-protected amino α, β-unsaturated ester of the formula 2(a) and each R³ is independently an amine protecting group as described hereinafter, and (2) where X is S, the α, β-unsaturated aldehyde of formula 1 is reacted with the trans-γ-protected mercapto α, β-unsaturated ester of the formula 2(b) and R³ is absent;
R⁴ and R⁴′ are the same or different and are an optionally substituted aryl group or an optionally substituted aralkyl group; and
R is an optionally substituted C₁-C₂₀ alkyl, alkenyl or alkynyl (“hydrocarbyl”) group, or is a compound of the formula 5:

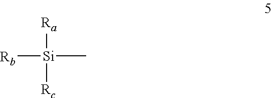

wherein Rₐ, R_{b}, and R_{c} are the same or different and are selected independently from the group consisting of an optionally substituted C₁-C₂₀ alkyl, alkenyl or alkynyl (“hydrocarbyl”) group, an alkoxy group, an optionally substituted aryl group, or an optionally substituted aralkyl group, or in preferred embodiments R is selected from the group consisting of trimethylsilyl (TMS), tert-butyldimethylsilyl (TBS), t-butyldimethylsilyl (TBDMS), t-butyldiphenylsilyl (TBDPS), triethylsilyl (TES), tert-butyldimethylsilyloxymethyl (TOM), triisopropylsilyl (TIPS), bis(trimethylsilyl)acetamide (BSA), triphenylsilyl, tetraisopropyldisiloxan-1,3-diyl, pivaloyl, acetyl (Ac), benzoyl (Bz), benzyl (Bn), β-Methoxyethoxymethyl ether (MEM), trityl (triphenylmethyl), methoxytrityl, dimethoxytrityl (bis-(4 methoxyphenyl)phenylmethyl) (DMT), methoxytrityl (4-methoxyphenyl diphenylmethyl (MMT)) methoxymethyl ether (MOM), p-methoxybenzyl ether (PMB), methylthiomethyl ether, tetrahydropyranyl (THP), a methyl ether, and an ethoxyethyl ether;
and wherein the concentration of the chiral ether catalyst of formula 4(a) or formula 4(b) in the reaction medium is between about 5 mole percent to about 30 mole percent and the process is done one-pot.

Processes of the invention achieve excellent levels of enantioselectivities (e.g. between about 90% to greater than about 99%, more preferably between about 95% to greater than about 99%, even more preferably greater than 96% to greater than 99%) and high levels of diastereoselectivities (e.g. between about 7:1 to greater than about 35:1 dr, in some embodiments between about 10:1 to greater than about 30:1 dr, in some embodiments between about 15:1 to greater than about 30:1 dr, and in some embodiments between about 20:1 to greater than about 30:1 dr).

Where X is N, R³ is an amine protecting group and can be any group that is used to protect at least one —NH— moiety by replacement of hydrogen and that is relatively inert to reaction conditions under which a nitrile is reduced. In a preferred embodiment, R³ may be linear or cyclic and may, for example, include or constitute one or more of the following amine protecting groups: tosyl (p-toluenesulfonyl) (Ts), p-Methoxybenzyl carbonyl (Moz or MeOZ), 9-Fluorenylmethyloxycarbonyl (FMOC), benzyl (Bn), p-Methoxybenzyl (PMB) 3,4-dimethoxybenzyl (DMPM), p-methoxyphenyl (PMP), a sulfonamide (e.g. a nosyl or nps group), tert-butyloxycarbonyl (BOC group), a carbobenzyloxy (CBZ group), a 2,4-nitrophenylsulfonyl group, a 2-nitrophenylsulfonyl group and a 4-nitophenyl sulfonyl group.

The weak base and solvent are defined hereinafter. Chlorinated solvents such as methylene chloride (CH₂Cl₂), and a weak base such as sodium acetate (NaOAc), are preferred.

In preferred embodiment of the invention where X is N and n is 1, the chiral ether compound of formula 4(a) or formula 4(b) is a chiral diarylprolinol ether catalyst that serves as a sterically bulky catalyst that effectively participates in iminium activation with enals and that creates high stereo-control.

In a preferred embodiment:

### n is 1 and X is N;

R⁴ and R⁴′ are the same or different and are a phenyl group which is optionally substituted with up to four substituents which are preferably selected from the group consisting of hydroxyl (OH), halogen (preferably F or Cl), CN, NO₂, a C₁-C₆ optionally substituted alkyl group (preferably CH₃, CH₂CH₃ or CF₃), a C₁-C₆ alkoxy group (which group may contain an unsaturated hydrocarbon), or a C₂-C₆ ether group (which group may contain an unsaturated hydrocarbon); and
R is selected from the group consisting of trimethylsilyl (TMS), tert-butyldimethylsilyl (TBS), and triethylsilyl (TES).

In another preferred embodiment, the invention provides a process for synthesizing a compound of the formula 3(a) by reacting an α, β-unsaturated aldehyde of formula 1 with a trans-γ-protected amino α, β-unsaturated ester of the formula 2(a) in a reaction medium comprising a weak base, a solvent, and a chiral diarylprolinol ether catalyst of the formula 4(a), wherein:

### n is 1 and X is N;

R¹ is a phenyl group which is optionally substituted with up to four substituents which are preferably selected from the group consisting of hydroxyl (OH), halogen (preferably F or Cl), CN, NO₂, a C₁-C₆ optionally substituted alkyl group (preferably CH₃, CH₂CH₃ or CF₃), a C₁-C₆ alkoxy group (which group may contain an unsaturated hydrocarbon), or a C₂-C₆ ether group (which group may contain an unsaturated hydrocarbon);
R² is an optionally substituted C₁-C₂₀ alkyl, alkenyl or alkynyl (“hydrocarbyl”) group;
R³ is tosyl (p-toluenesulfonyl) (Ts);
R⁴ and R⁴′ are the same or different and are a phenyl group which is optionally substituted with up to four substituents which are preferably selected from the group consisting of hydroxyl (OH), halogen (preferably F or Cl), CN, NO₂, a C₁-C₆ optionally substituted alkyl group (preferably CH₃, CH₂CH₃ or CF₃), a C₁-C₆ alkoxy group (which group may contain an unsaturated hydrocarbon), or a C₂-C₆ ether group (which group may contain an unsaturated hydrocarbon);
R is selected from the group consisting of trimethylsilyl (TMS), t-butyldimethylsilyl (TBDMS), t-butyldiphenylsilyl (TBDPS), triethylsilyl (TES), tert-butyldimethylsilyloxymethyl (TOM), triisopropylsilyl (TIPS), bis(trimethylsilyl)acetamide (BSA), triphenylsilyl, and tetraisopropyldisiloxan-1,3-diyl;
the enantioselectivity of the process is between about 96% to greater than about 99% and wherein the concentration of the chiral diarylprolinol ether catalyst of formula 4(a) in the reaction medium is between about 10 mole percent to about 20 mole percent.

In a particularly preferred embodiment, the invention provides a process for synthesizing a compound of the formula 3(a) by reacting an α, β-unsaturated aldehyde of formula 1 with a trans-γ-protected amino α, β-unsaturated ester of the formula 2(a) in a reaction medium comprising a weak base, a solvent, and a chiral diarylprolinol ether catalyst of the formula 4(a), wherein:

### n is 1 and X is N;

R¹ is a phenyl group which is optionally substituted with up to four substituents which are preferably selected from the group consisting of hydroxyl (OH), halogen (preferably F or Cl), CN, NO₂, a C₁-C₆ optionally substituted alkyl group (preferably CH₃, CH₂CH₃ or CF₃), a C₁-C₆ alkoxy group (which group may contain an unsaturated hydrocarbon), or a C₂-C₆ ether group (which group may contain an unsaturated hydrocarbon);
R² is an optionally substituted C₁-C₂₀ alkyl, alkenyl or alkynyl (“hydrocarbyl”) group;
R³ is tosyl (p-toluenesulfonyl) (Ts);
R⁴ and R⁴′ are a phenyl group;
R is selected from the group consisting of trimethylsilyl (TMS), tert-butyldimethylsilyl (TBS), and triethylsilyl (TES);
the solvent is methylene chloride (CH₂Cl₂), the weak base is sodium acetate (NaOAc), the enantioselectivity of the process is between about 96% to greater than about 99%, and the concentration in the reaction medium of the chiral diarylprolinol ether catalyst of formula 4(a) is about 20 mole %.

In another embodiment of the invention, compounds of the formula 3(a) or formula 3(b) are synthesized as described above and, where X is N, are deprotected by removal of the group R³ as described hereinafter.

Catalysts of the invention have the formula 4(a) or formula 4(b) as described above.

The highly-functionalized, trisubstituted chiral pyrrolidines, piperidines, tetrahydrothiophenes, and thianes made by processes of the invention may exhibit pharmacological activity and are useful as intermediates in the manufacture of naturally occurring substances and synthetic biologically active compositions.

Thus, syntheses of the invention represent novel organocatalytic cascade Michael-Michael processes that directly convert simple achiral substrates to highly fuctionalized trisubstituted chiral pyrrolidines, piperidines, tetrahydrothiophenes, and thianes with high levels of enantio- and diastereoselectivity (8-10). The reactions are accomplished with remarkable efficiency, e.g. where n is 1 and X is N, by use of a simple chiral diphenylprolinol silyl ether as a promoter. This one-pot transformation produces a complex molecular architecture formed with high stereo-control of three new stereogenic centers and an array of exploitable orthogonal functionality for further synthetic elaboration.

These and other aspects are described further in the detailed description of the invention.

### BRIEF DESCRIPTION OF THE FIGURES

FIG. 1 illustrates a proposed mechanism for an asymmetric Michael-Michael process promoted by an organocatalyst in accordance with the invention.

FIG. 2 illustrates the X-ray crystal structure analysis of compound 3c based on its derivative 4.

### DETAILED DESCRIPTION OF THE INVENTION

As used herein, the following terms are used to describe the present invention. The definitions provided below, within context, may be used exclusively, or may be used to supplement definitions which are generally known to those of ordinary skill in the art.

Unless otherwise indicated, the present invention is not limited to particular molecular structures, substituents, synthetic methods, reaction conditions, or the like, and accordingly, these may vary. It is also to be understood that the terminology used herein is for the purpose of describing particular embodiments only and is not intended to be limiting.

As used in the specification and the attached claims, the use of “a,” “an” and “the” include references to plural subject matter referred to unless the context clearly dictates otherwise. Thus, for example, reference to “a catalyst” includes a single catalyst as well as a combination or mixture of two or more catalysts, reference to “a reactant” encompasses a combination or mixture of different reactants as well as a single reactant, and the like.

A term which is subsumed under another term may be embraced by the broader term or by the more narrow specific term as appropriate within the context of the use of that term. All terms used to describe the present invention are used within context.

In this specification and in the claims that follow, reference will be made to a number of terms, which shall be defined to have the following meanings:

As used herein, the phrase “according to the formula”, “having the formula” or “having the stricture” is not intended to be limiting and is used in the same way that the term “comprising” is commonly used. The term “independently” is used herein to indicate that the recited elements, e.g., R groups or the like, can be identical or different.

“Optional” or “optionally” means that the subsequently described circumstance may or may not occur, so that the description includes instances where the circumstance occurs and instances where it does not. For example, the term “optionally substituted” means that a non-hydrogen substituent may or may not be present on a given atom, and, thus, the description includes structures where a non-hydrogen substituent is present and structures where a non-hydrogen substituent is not present.

The term “compound” is used herein to refer to any specific chemical compound disclosed herein. Within its use in context, the term generally refers to a single compound, such as a single enantiomer or diastereomer, but in certain instances may also refer to stereoisomers and/or optical isomers (including racemic mixtures) of disclosed compounds.

The term “effective” is used in context to describe an amount of a compound, component, condition or other aspect of the invention which occurs in an amount or at a level which is sufficient to effect an intended result, whether that compound, component or condition is an organocatalyst according to the present invention, a solvent, a reactant, an amount of heat or other aspect of the invention.

The term “alkyl” as used herein refers to a branched or unbranched saturated hydrocarbon group typically although not necessarily containing 1 to about 20 carbon atoms, such as methyl, ethyl, n-propyl, isopropyl, n-butyl, isobutyl, t-butyl, octyl, decyl, and the like, as well as cycloalkyl (“carbocyclic”) groups such as cyclopentyl, cyclohexyl and the like. Generally, although again not necessarily, alkyl groups herein contain 1 to about 20 carbon atoms, preferably 1 to about 12 carbon atoms, more preferably about 1 to 6 carbon atoms (“lower alkyl”). “Substituted alkyl” refers to alkyl substituted with one or more substituent groups as otherwise described herein, and subsumes the terms “heteroatom-containing alkyl” or “heteroalkyl” which, in context, refer to an alkyl substituent in which at least one carbon atom is replaced with a heteroatom, such as an ether group, thioether group, a pyrrole or piperidine, as described in further detail infra. If not otherwise indicated, the terms “alkyl” and “lower alkyl” include linear, branched, cyclic, unsubstituted, substituted alkyl groups, respectively.

The term “alkenyl” as used herein refers to a linear, branched or cyclic hydrocarbon group of 2 to about 20 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein contain 2 to about 20 carbon atoms, preferably 2 to 6 carbon atoms. The term “lower alkenyl” describes an alkenyl group of 2 to 6 carbon atoms. The term “substituted alkenyl” refers to alkenyl substituted with one or more substituent groups, and subsumes the term “heteroatom-containing alkenyl” and “heteroalkenyl” which refer to alkenyl in which at least one carbon atom is replaced with a heteroatom. If not otherwise indicated, the terms “alkenyl” and “lower alkenyl” include linear, branched, cyclic and unsubstituted or substituted alkenyl and lower alkenyl, respectively. Note that alkenyl groups are used within context and not where a reaction scheme would dictate that its use is unfavorable.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein contain 2 to about 18 carbon atoms, preferably 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively. Note that alkynyl groups are used within context and not where a reaction scheme would dictate that its use is unfavorable.

The term “alkoxy” as used herein intends an alkyl group bound through a single, terminal ether linkage; that is, an “alkoxy” group may be represented as —O-alkyl where alkyl is as defined above. A “lower alkoxy” group describes an alkoxy group containing 1 to 6 carbon atoms, and includes, for example, methoxy, ethoxy, n-propoxy, isopropoxy, t-butyloxy, etc. Preferred substituents falling within “C₁-C₆ alkoxy” or “lower alkoxy” herein contain 1 to 4 carbon atoms, and additionally preferred such substituents contain 1 or 2 carbon atoms (i.e., methoxy and ethoxy).

The term “aryl” as used herein, and unless otherwise specified, refers to an aromatic group generally containing 5 to 30 carbon atoms and containing a single aromatic ring (phenyl) or multiple aromatic rings that are fused together, directly linked, or indirectly linked (such that the different aromatic rings are bound to a common group such as a methylene or ethylene moiety). Preferred aryl groups contain 5 to 20 carbon atoms, and particularly preferred aryl groups contain 5 to 12 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” are subsumed under the term aryl, in which at least one carbon atom of a carbocyclic aryl group is replaced with a heteroatom. If not otherwise indicated, the term “aryl” includes unsubstituted, substituted, and/or heteroatom-containing aromatic groups.

The term “carbocyclic” refers to a cyclic ring structure, which, in context, is saturated or unsaturated and contains exclusively carbon atoms within the ring structure. The term “heterocyclic” refers to a cyclic ring structure, which, in context, is either saturated or unsaturated and may contain one or more atoms other than carbon atoms (e.g., N, O, S, etc.) within the ring structure.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms, while preferred aralkyl and alkaryl groups contain 6 to 20 carbon atoms, and particularly preferred such groups contain 6 to 12 carbon atoms.

The term “amino” is used herein to refer to the group —NZ¹Z² wherein Z¹ and Z² are hydrogen or nonhydrogen substituents, with nonhydrogen substituents including, for example, alkyl, aryl, alkenyl, aralkyl, and substituted and/or heteroatom-containing variants thereof, as otherwise specifically described in the specification.

The terms “halogen” and related terms such as “halo” are used in the conventional sense to refer to a chloro, bromo, fluoro or iodo substituent.

The term “heteroatom-containing” as in a “heteroatom-containing alkyl group”, (also termed a “heteroalkyl” group), “heterocyclic” group or a “heteroatom-containing aryl group” (also termed a “heteroaryl” group) refers to a molecule, group, linkage or substituent in which one or more carbon atoms are replaced with an atom other than carbon, e.g., nitrogen, oxygen, sulfur, phosphorus or silicon, preferably from 1 to 3 nitrogen, oxygen or sulfur atoms. Similarly, the term “heteroalkyl” refers to an alkyl group that is heteroatom-containing, the term “heterocyclic” more broadly refers to a cyclic group that is heteroatom-containing (thus, also potentially containing unsaturated groups), the terms “heteroaryl” and heteroaromatic” respectively refer to “aryl” and “aromatic” groups that are heteroatom-containing, and the like. Examples of heteroalkyl groups include alkoxyaryl, alkylsulfanyl-substituted alkyl, N-alkylated amino alkyl, and the like. Examples of heterocyclic groups, include heteroaryl groups such as pyrrolyl, pyrrolidinyl, pyridinyl, quinolinyl, indolyl, pyrimidinyl, imidazolyl, 1,2,4-triazolyl, tetrazolyl, thiazole, etc., and heteroatom-containing alicyclic groups such as pyrrolidino, morpholino, piperazino, piperidino, etc.

“Hydrocarbyl” refers to univalent hydrocarbyl radicals containing 1 to about 30 carbon atoms, preferably 1 to about 20 carbon atoms, preferably about 1 to 12 carbon atoms, preferably about 1 to 6 carbon atoms, including linear, branched, cyclic, saturated and unsaturated species, such as alkyl groups, alkenyl groups, aryl groups, and the like. “Substituted hydrocarbyl” or “optionally substituted hydrocarbyl” refers to hydrocarbyl substituted with one or more substituent groups, and the term “heteroatom-containing hydrocarbyl” is subsumed under the term hydrocarbyl in which at least one carbon atom is replaced with a heteroatom. Unless otherwise indicated in context, the term “substituted hydrocarbyl” is to be interpreted as including substituted and/or heteroatom-containing hydrocarbyl moieties, including heterocyclic moieties.

The term “substituted” as in “substituted alkyl,” “substituted aryl,” “substituted hydrocarbyl”, etc. and the like, as described hereinabove, refers to a carbon-containing or other moiety used in context, such as hydrocarbyl, alkyl, aryl, including cyclic versions of same, or other moiety, at least one hydrogen atom bound to a carbon (or other) atom is replaced with one or more non-hydrogen substituents. Examples of such substituents include, without limitation: functional groups such as halo, hydroxyl, sulfhydryl, C₁-C₂₄ alkoxy, C₂-C₂₄ alkynyloxy, C₂-C₂₄ alkynyloxy, C₅-C₂₀ acyloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO), carbamoyl (—(CO)—NH₂), mono-substituted C₁-C₂₄ alkylcarbamoyl (—(CO)—NH(C₁-C₂₄ alkyl)), di-substituted alkylcarbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N⁺≡C—), cyanato (—O—C≡N), isocyanato (—O—N^{+≡}C⁻), isothiocyanato (—S—C≡N), azido (—N═N⁺≡N⁻), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₅-C₂₀ arylamido (—NH—(CO)-aryl), imino (—CR═NH where R=hydrogen, C₁-C₂₄ alkyl, C₅-C₂₀ aryl, C₆-C₂₀ alkaryl, C₆-C₂₀ aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO—OH), sulfonato (—SO₂O—), C₁-C₂₄ alkylsulfanyl (—S-alkyl; also termed “alkylthio”), arylsulfanyl (—S-aryl; also termed “arylthio”), C₁-C₂₄ alkylsulfinyl —(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O⁻)), phospho (—PO₂), and phosphino (—PH₂); and the hydrocarbyl moieties C₁-C₂₄ alkyl (preferably, C₁-C₂₀ alkyl, more preferably C₁-C₁₂ alkyl, most preferably C₁-C₆ alkyl), C₂-C₂₄ alkenyl (preferably C₂-C₂₀ alkenyl, more preferably C₂-C₁₂ alkenyl, most preferably C₂-C₆ alkenyl), C₂-C₂₄ alkynyl (preferably C₂-C₂₀ alkynyl, more preferably C₂-C₁₂ alkynyl, most preferably C₂-C₆ alkynyl), C₅-C₃₀ aryl (preferably C₅-C₂₀ aryl, more preferably C₅-C₁₂ aryl), and C₆-C₃₀ aralkyl (preferably C₆-C₂₀ aralkyl, more preferably C₆-C₁₂ aralkyl). In addition, the aforementioned functional groups may, if a particular group permits within the context of a reaction pathway or synthesis, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated. Substitutions which are cyclic groups may be bonded to a single atom within a moiety or more than one substituent may be joined to form a cyclic ring, thus forming for example, bi- or tricyclic groups.

When the term “substituted” appears prior to a list of possible substituted groups, it is intended that the term apply to every member of that group. For example, the phrase “substituted alkyl and aryl” or “substituted alkyl or aryl” is to be interpreted as “substituted alkyl and substituted aryl” or “substituted alkyl or substituted aryl.”

The term “chiral” refers to a structure that does not have an improper rotation axis (Sₙ), i.e., it belongs to point group Cₙ or Dₙ. Such molecules are thus chiral with respect to an axis, plane or center of asymmetry. Preferred “chiral” molecules herein are in enantiomerically pure form, such that a particular chiral molecule represents at least about 95 wt. % (95% ee) of the composition in which it is contained, more preferably at least about 99 wt. % (99% ee) of that composition, more preferably about 99+wt. % (99+% ee) of that composition.

The term “enantioselective” refers to a chemical reaction that preferentially results in one enantiomer relative to a second enantiomer, i.e., gives rise to a product of which a desired enantiomer represents at least about 50 wt. %. Preferably, in the enantioselective reactions herein, the desired enantiomer represents at least about 65 wt. % (65% enantiomeric enrichment or “ee”) of the product, preferably at least about 75 wt. % (75% ee), at least about 85 wt. % (85% ee), at least about 95 wt. % (95% ee), at least 99 wt. % (99% ee) and at least 99+st. % (99+% ee) of the product.

The term “temperature” is generally used to describe the temperature at which a reaction takes place. In general, reactions according to the present invention may take place at a temperature ranging from significantly below room temperature (e.g., −78° C.) or above temperature (for example, at reflux temperatures which, depending on the boiling point of the solvent used, can be several hundred degrees Celsius), but preferably reactions proceed at or about ambient or room temperature (i.e., the temperature of the surrounding laboratory or manufacturing facility).

The term “solvent” is used to describe a medium (typically, but not necessarily inert) in which a reaction takes place using the catalysts as described herein. Solvents may include polar and non-polar solvents, e.g. halogenated solvents (preferably the chlorinated solvents trichlorethylene, perchlorethylene (tetrachlorethylene), methylene chloride (CH₂Cl₂), carbon tetrachloride (CCl₄)), chloroform (CHCl₃), 1,1,1-trichloroethane (methyl chloroform, CH₃—CCl), and most preferably methylene chloride), diethyl ether, H₂O, pyridine, triethanolamine, tetrahydrofuran, 1,4-dioxane, acetonitrile, dimethylacetamide (DMA), dimethylformamide (DMF), dimethylsulfoxide (DMSO), acetonitrile, nitromethane, methanol, ethanol, isopropanol, aqueous alcohol (methanol, ethanol, isopropanol, N-methylpyrrolidone (NMP), ethylacetate, benzene, toluene, tetrahydrofuran and mixtures thereof.

The term “acid” is used (within the context of its use) as it is typically understood by those of ordinary skill in the art to describe a protic acid (proton donor) or Lewis acid for use in the present invention and may include strong acids, such as hydrochloric acid, sulfuric acid, phosphoric acid, sulfamic acid, etc., organic acids, such as acetic acid, benzoic acid, mandelic acid, propionic acid and butyric acid, etc. and a number of Lewis acids well-known in the art, such as AlX₃, BX₃, FeX₃, GaX₃, SbX₃, SnX₄, ZnX₃, where X is a halogen atom or an inorganic radical, among numerous others.

The term “base” is used (within the context of its use) as it is typically understood by those of ordinary skill in the art to describe a proton acceptor or Lewis base. Lewis bases are electron acceptors which are well-known in the art and include such bases as NH₃, PF₃, PCl₃, H₂S, H₂O, HOCH₂CH₂CH₂OH, Cl⁻, OH⁻, O₂CCO₂²⁻, and any negatively charged ion.

Processes of the inventions can be carried out in weak bases such as the ammonium, transition metal, alkali metal and alkali earth metal salts of HCOO⁻, AcO⁻, CF₃COO⁻, tBuCOO⁻, HCO₃⁻. HSO₄²⁻, HSO₃⁻, H₂₂PO₃⁻, HPO₃²⁻ and phenolates such as 2,4-dinitrophenolate. For example, bases such as NaOAc, NaHCO₃, triethylamine (TEA), a tertiary amine base like DIEA (N,N-diisopropylethyl amine), tetraethyl-ammonium chloride, and sodium fort hate can be used.

Those of ordinary skill in the art are able to select and vary reaction conditions as necessary to perform the reactions described herein. For example, the reaction conditions specified in Tables 1 and 2 hereinafter provide guidelines as to the relative amounts of the weak base, solvent, and chiral diarylprolinol ether catalyst and these amounts may be reasonably varied to account for reaction medium compositions or conditions. The concentration of the chiral diarylprolinol ether catalyst of formula 4(a) or formula 4(b) in the reaction medium is between about 5 mole percent to about 30 mole percent, more preferably between about 7 mole percent to about 25 mole percent, even more preferably between about 10 mole percent to about 20 mole percent. Unless specified to the contrary, the reactions described herein take place at atmospheric pressure within a temperature range from 5° C. to 170° C. (preferably from 10° C. to 50° C.; most preferably at “room” or “ambient” temperature, e.g., about 20°-30° C.). However, there are some reactions where the temperature range used in the chemical reaction will be above or below these temperature ranges. Further, unless otherwise specified, the reaction times and conditions are intended to be approximate, e.g., taking place at about atmospheric pressure within a temperature range of about 5° C. to about 100° C. (preferably from about 10° C. to about 50° C.; most preferably about 20°-30° C.) over a period of about 1 to about 100 hours (preferably about 5 to 60 hours).

The term “protecting group” refers to a chemical moiety or group which protects or prevents an active moiety or group from participating with or interfering with one or more chemical synthetic steps and its removal restores the moiety to its original active state. The term protecting group as used herein refers to those groups intended to protect against undesirable reactions during synthetic procedures. One of ordinary skill in the art can readily choose an appropriate protecting group to facilitate synthetic reactions according to method aspects of the present invention without engaging in undue experimentation. See e.g. “Protecting Groups in Organic Synthesis”, 3^{rd} Edition”, by Philip J. Kocienski or “Greene's Protective Groups in Organic Synthesis”, 4^{th} Edition” by Peter G. M. Wuts and Theodora W. Greene. The protecting group group R³ has been defined above.

Certain embodiments of the invention in which X is N involve the step of deprotecting compounds of the formula 3(a) or formula 3(b) to remove the group R³. One of ordinary skill in the art can readily choose an appropriate deprotecting reaction without engaging in undue experimentation. See “Greene's Protective Groups in Organic Synthesis”, 4^{th} Edition” by Peter G. M. Wuts and Theodora W. Greene.

“Amine protecting group” has been defined in the description of R³ provided above in the Summary of the Invention (i.e. an amine protecting group can be any group that is used to protect at least one —NH— moiety by replacement of hydrogen and that is relatively inert to reaction conditions under which a nitrile is reduced).

For example, R³ includes or constitutes tosyl (p-toluenesulfonyl)(Ts), deprotection may be accomplished using concentrated acid (HBr, H₂SO₄) and strong reducing agents (e.g. sodium in liquid ammonia, sodium napthalene) or by reaction with cesium carbonate in THF-MeOH; where R³ includes or constitutes p-methoxybenzyl carbonyl (Moz or MeOZ), deprotection may be accomplished by hydrogenolysis; where R³ includes or constitutes 9-fluorenylmethyloxycarbonyl (FMOC), deprotection may be accomplished using a base such as piperidine; where R³ includes or constitutes benzyl (Bn), deprotection may be accomplished by hydrogenolysis; where R³ includes or constitutes p-methoxybenzyl (PMB), deprotection may be accomplished by hydrogenolysis; where R³ includes or constitutes 3,4-dimethoxybenzyl (DMPM), deprotection may be accomplished by hydrogenolysis; where R³ includes or constitutes p-methoxyphenyl (PMP), deprotection may be accomplished by using ammonium cerium(TV) nitrate (CAN); where R³ includes or constitutes a sulfonamide (e.g. a nosyl or nps group), deprotection may be accomplished by using samarium iodide and tributyltin hydride, where R³ includes or constitutes BOC, deprotection may be accomplished by concentrated, strong acid. (such as HCl or CF₃COOH); and where R³ includes or constitutes CBZ, deprotection may be accomplished by hydrogenolysis.

The term “isolation” or “isolating” refers to the process or method by which a product compound or composition is isolated from a reaction mixture. These methods may include various forms of chromatography, including those which employ chiral packing or support in columns, including standard column chromatography, medium and high pressure liquid chromatography, crystallization, precipitation, etc., countercurrent distribution, etc. All methods for isolating compounds according to the present invention are well know in the art.

In the molecular structures herein, the use of bold and dashed lines to denote particular conformation of groups follows the IUPAC convention. A bond indicated by a broken line indicates that the group in question is below the general plane of the molecule as drawn (the “α” configuration), and a bond indicated by a bold line indicates that the group at the position in question is above the general plane of the molecule as drawn (the “β” configuration).

The invention is illustrated further in the following experimental section and examples, which are illustrative and in no way limiting.

### Experimental Section

### Overview

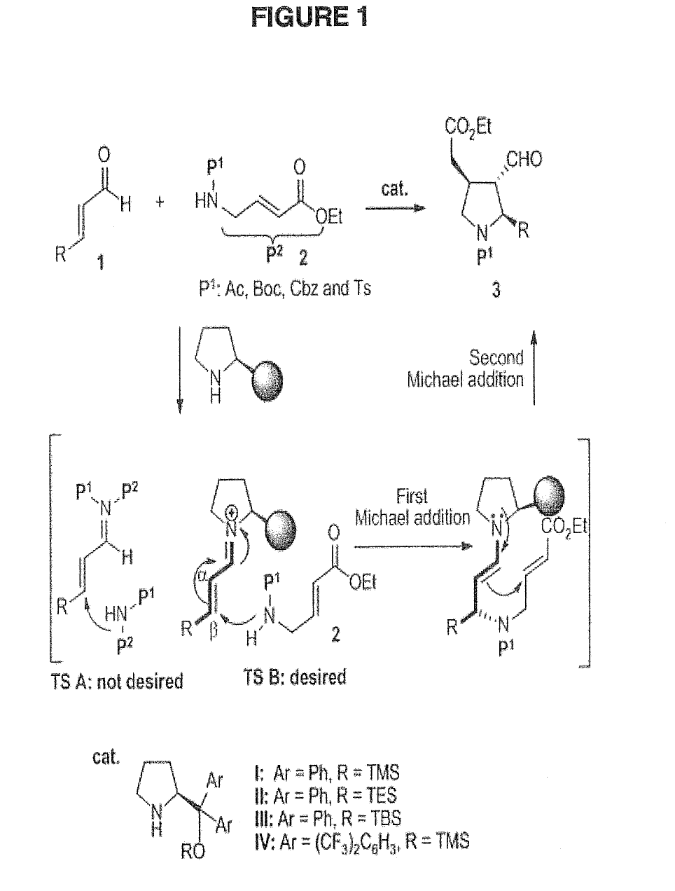

Central to the implementation of the novel cascade Michael-Michael reactions described herein was the recognition of several reactivity and selectivity issues that had to be addressed (FIG. 1). First, although a “N”-centered nucleophile has been employed for conjugate addition reactions, the more active specific species was required as a result of its weak nucleophilicity. (11) Second, the amine in the trans-γ-protected amino α, β-unsaturated ester of formula 2 should not function as catalyst for the formation of imminium (see FIG. 1 TS A), and must serve as nucleophile for conjugate addition to the catalyst activated imminium (see FIG. 1 TS B). Third, the substrate amine ester of formula 2 should be stable during the reaction without undergoing intramolecular lactamization. Consequently, in considering these parameters, we designed trans 4-amino protected α, β-unsaturated ester of formula 2. The protected amino group (e.g. amides, carbamates or sulfonamides) prevented formation of an iminium with enals of formula 1, while the enhanced acidity rendered compounds of formula 2 more readily deprotonated under basic conditions to produce a more nucleophilic nitrogen anion for the first Michael addition reaction. Moreover, the trans-geometry could significantly reduce the intramolecular lactamization. With the respect to the catalysts, we reasoned that sterically bulky catalysts such as chiral diarylprolinol ethers I-IV (see FIG. 1) would prove useful due to their established capacity to effectively participate in iminium activation with enals and create high stereo-control. (12).

### Example 1

### Orienting Experiments

Referring to Table 1 below, our orienting experiments were performed by reacting trans-cinnamaldehyde 1a with trans γ-N-protected α, β-unsaturated esters 2 in the presence of diphenylprolinol silyl ether catalyst I and NaOAc as base (Table 1). The preliminary studies revealed that the reaction efficiency highly depended on the protection form of the nitrogen nucleophile (entries 1-4). No reaction occurred with Ac, Boc and Cbz whereas a promising result was obtained when Ts was used. This indicated that the nucleophilicity of the nitrogen was critical for the cascade process. The strong electron-withdrawing capacity of Ts group rendered the NH more acidic and thus readily generating more nucleophilic nitrogen anion under a basic condition. Notably, we did not observe the lactamization reaction in 2. The subsequent survey of different chiral organocatalysts disclosed that similar results were achieved with catalysts II and III (Table 1, entries 5-6). However, the process proceeded very poorly with IV (Table 1, entry 7).

TABLE 1
Optimization of organocatalytic enantioselective aza-
Michael-Michael addition reactions^{a}

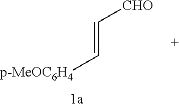

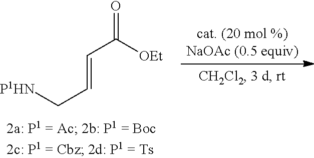

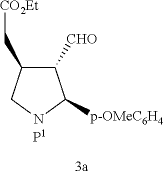

| Entry | Cat | P¹ | Yield (%)^{b} | ee (%)^{c} | dr^{d} |
|---|---|---|---|---|---|
| 1 | I | Ac | 0 | ND^{e} | ND^{e} |
| 2 | I | Boc | 0 | ND^{e} | ND^{e} |
| 3 | I | Cbz | 0 | ND^{e} | ND^{e} |
| 4 | I | Ts | 83 | 93 | 15:1 |
| 5 | II | Ts | 78 | 91 | 13:1 |
| 6 | III | Ts | 75 | 87 | 14:1 |
| 7 | IV | Ts | <5 | ND^{e} | ND^{e} |

^{a}Unless otherwise specified, to a solution of trans-4-methoxycinna-maldehyde 1a (32 mg, 0.2 mmol) in the presence of catalyst (20 mol %) and NaOAc (8.2 mg, 0.1 mmol) in CH₂Cl₂ (0.5 mL) was added 2 (0.1 mmol) and the resulting solution was stirred for 3 d at rt.
^{b}Isolated yield.
^{c}Determined by chiral HPLC analysis (Chiralcel OD-H).
^{d}Determined by ¹H NMR.
^{e}Not determined.

### Example 2

### Optimization of Reaction Conditions

In further optimization of reaction conditions, we focused on varying reaction parameters including base, solvent and catalyst loading (see Supporting Information for detail). In these experiments, the optimal results with respect to reaction time, yields, enantio- and diastereoselectivity of the Michael-Michael reaction were obtained when the reaction cascade was performed with 10 mol % I in CHCl₃ using 1.0 equiv. of NaOAc.

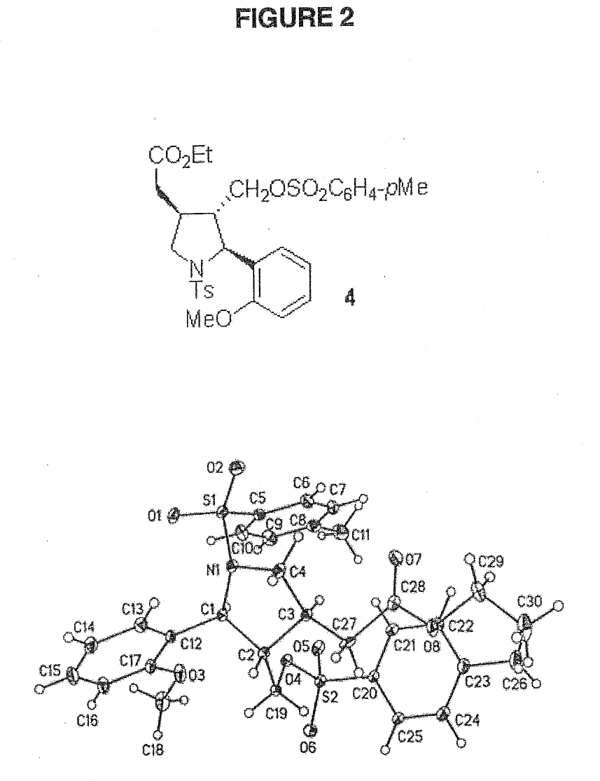

As revealed in Table 2, the cascade process serves as a general approach to the preparation of highly functionalized trisubstituted chiral pyrrolidines. Significantly, three new stereogenic centers are created in a one-pot transformation in high yields (80-94%) and with excellent levels of enantioselectivities (96->99% ee) and high diastereoselectivities (7:1 to >30:1 dr). Significant structural variation of α, β-unsaturated aldehydes can be tolerated. The electronic nature of the aryl rings of α, β-unsaturated aldehydes 1 has apparently limited influence on the stereochemical outcome. The reaction system is inert to the electronic effect, as evidenced that in all cases, extremely high enantioselectivity (96->99% ee) and high diastereoselectvity (up to >30:1) are observed regardless of electron-donating (entries 1-3), neutral (entry 4), combination of electron-donating and withdrawing (entry 5) and withdrawing (entries 6-11) substituents tested. Probing the steric effect on the enantio- and diasteroselectivity of the cascade processes indicates that such impact is also minimal (96% ee, 7.5:1 dr, entry 3). The relative and absolute configuration of product 3c is determined by the X-ray crystal structural analysis based on its derivative 4 (FIG. 2). (13)

TABLE 2
Catalyst I promoted cascade aza-Michael-Michael reactions
for one-pot synthesis of chiral pyrrolidines^{a}

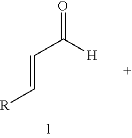

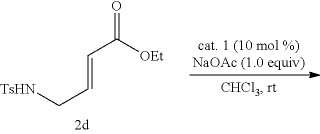

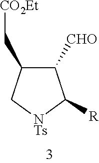

| Entry | R | 3 | t/d | Yield (%)^{b} | ee (%)^{c} | dr^{d} |
|---|---|---|---|---|---|---|
| 1 | 4-MeOC₆H₄ | 3a | 4 | 92 | >99^{e} | >30:1 |
| 2 | 3-MeOC₆H₄ | 3b | 3 | 91 | >99^{e} | 30:1 |
| 3 | 2-MeOC₆H₄ | 3c | 4 | 90 | 96 | 7.5:1 |
| 4 | Ph | 3d | 3 | 94 | >99 | 21:1 |
| 5 | 3-MeO-4-AcC₆H₃ | 3e | 4 | 88 | 96^{e} | 27:1 |
| 6 | 4-CF₃C₆H₄ | 3f | 4 | 92 | >99 | 12:1 |
| 7 | 4-NO₂C₆H₄ | 3g | 4 | 85 | >99 | 10:1 |
| 8 | 3-NO₂C₆H₄ | 3h | 3 | 80 | >99 | 7:1 |
| 9 | 4-CNC₆H₄ | 3i | 3 | 89 | >99 | >30:1 |
| 10 | 4-FC₆H₄ | 3j | 4 | 85 | >99^{e} | 14:1 |
| 11 | 4-BrC₆H₄ | 3k | 4 | 90 | >99 | 18:1 |

^{a}Reaction conditions: unless specified, see footnote a in Table 1.
^{b}Isolated yields.
^{c}Determined by chiral HPLC analysis (Chiralpak AS-H, AD, OJ-H, or Chiralcel OD-H).
^{d}Determined by ¹H NMR.
^{e}Converted to enone via reacting product aldehyde with Ph₃P—CHCOPh for chiral HPLC analysis.

### Discussion of Experimental Results

Driven by the lack of an efficient method for the preparation of synthetically significant trisubstituted chiral piperidines, tetrahydrothiophenes, and thianes, we have discovered unprecedented organocatalytic, highly enantioselective cascade Michael-Michael reactions. In one embodiment, the exemplified processes were efficiently catalyzed by readily available (5) diphenylprolinol TMS ether to give trisubstituted synthetically useful, highly functionalized chiral pyrrolidines. (We carried out a 1 g scale of reaction of 1a with 2d in the presence of catalyst I (10 mol %) and NaOAc (1.0 equiv) in CHCl₃ for 4d to afford 3a in 93% yield, >99% ee and >30:1 dr.) The scaffold serves as an efficient starting point for further synthetic manipulation in total synthesis of natural products, biologically significant therapeutics and the diversity oriented library synthesis. Moreover, in principle, the cascade strategy can be exploited for the synthesis of synthetically and medicinally important chiral piperidines, tetrahydrothiophenes, and thianes.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

### REFERENCES

1 Reviews of chemistry and biology of pyrrolidines, see: (a) F. J. Sardina, H. Rapoport, Chem. Rev., 1996, 96, 1825; (b) D. O'Hagan, Nat. Prod. Rep., 2000, 17, 435; (c) F. Bellina, R. Rossi, Tetrahedron, 2006, 62, 7213;
2. G. Marsicano, S. Goodenough, K. Monory, K. Science, 2003, 302, 84.
3 S. Banerjee, M. Hussain, Z. Wang, A. Saliganan, M. Che, D. Bonfil, M. Chem., F. H. Sarkar, Cancer Res., 2007, 67, 3818.
4 G. T. Wang, Y. Chen, S. Wang, R. Gentles, T. Sowin, W. Kati, S. Muchmore, V. Giranda, K. Stewart, H. Sham, D. Kempf, W. G. Layer, J. Med. Chem., 2001, 44, 1192.
5 M. M.-C. Lo, C. S, Neimann, S, Nagayama, E. O. Perlstein, S. L. Schreiber, J. Am. Chem. Soc., 2004, 126, 16077.
6 (a) D. Enders, T. Thiebes, Pure Appl. Chem., 2001, 73, 573; (b) F.-X. Felpin, J. Lebreton, Eur. J. Org. Chem., 2003, 3693; (c) S. Husinec, V. Savic, Tetrahedron: Asymmetry, 2005, 16, 2047; (d) I. Coldham, R. Hufton, Chem. Rev., 2005, 105, 2765; (e) G. Pandey, P. Banerjee, S. R. Gadre, Chem. Rev., 2006, 106, 4484; (f) V. Nair, T. D. Suja, Tetrahedron, 2007, 63, 12247.
7 (a) J. L. Vicario, S. Reboredo, D. Badia, L. Carrillo, Angew. Chem., Int. Ed., 2007, 46, 5168; (b) I. Ibrahem, I. R. Rioas, J. Vesely, A. Córdova, Tetrahedron. Lett., 2007, 48, 6252; (c) C. Guo, M.-X. Xue, M.-K. Zhu, L.-Z. Gong, Angew. Chem., Int. Ed., 2008, 47, 3414; (d) Chen, X.-H.; Zhang, W.-Q.; Gong, L.-Z. J. Am. Chem. Soc., 2008, 130, 5652.
8 Reviews of organocatalytic cascade reactions, see: (a) D. Enders, C. Grondal, M. R. M. Hüttl, Angew. Chem., Int. Ed., 2007, 46, 1570; (b) A. M. Walji, D. W. C. MacMillan, Synlett, 2007, 1477; (c) X.-H. Yu, Wang, W. Org. Biomol. Chem., 2008, 6, 2037.
9. Recent selected examples of organocatalytic cascade reactions, see: (a) D. B. Ramachary, N. S. Chowdari, C. F. Barbas, III, Angew. Chem. 2003, 115, 4265; D. B. Ramachary, N. S. Chowdari, C. F. Barbas, III, Angew. Chem. Int. Ed. 2003, 42, 4233; (b) D. B. Ramachary, K. Anebouselvy, N. S. Chowdari, C. F., Barbas, III. J. Org. Chem., 2004, 69, 5838.; (c) Y. Yamamoto, N. Momiyama, H. Yamamoto, J. Am. Chem. Soc., 2004, 126, 5962; (d) Y. Huang, A. M. Walji, C. H. Larsen, D. W. C. MacMillan, J. Am. Chem. Soc., 2005, 127, 15051; (e) J. W. Yang, M. T. H. Fonseca, B. List, J. Am. Chem. Soc., 2005, 127, 15036; (f) M. Marigo, T. Schulte, J. Franzen, K. A. Jøorgensen, J. Am. Chem. Soc., 2005, 127, 15710; (g) X.-F. Zhu, C. E. Henry, J. Wang, T. Dudding, O. Kwon, Org. Lett., 2005, 7, 138; (h) D. Enders, M. R. M. Hüttl, C. Grondal, G. Raabe, Nature 2006, 441, 861; (i) W. Wang, H. Li, J. Wang, L. Zu, J. Am. Chem. Soc., 2006, 128, 10354; (j) Y. Wang, X.-F. Liu, L. Deng, J. Am. Chem. Soc., 2006, 128, 3928; (k) D. Enders, M. R. M. Hüttl, J. Runsink, G. Raabe, B. Wendt, Angew. Chem., Int. Ed., 2007, 46, 467; (1) A. Carlone, S. Cabrera, M. Marigo, K. A. Jørgensen, Angew. Chem., Int. Ed., 2007, 47, 1101; (m) H. Li, J. Wang, H. Xie, L. Zu, W. Jiang, E. N. Duesler, W. Wang, Org. Lett., 2007, 9, 965; (n) H. Sunden, I. Ibrahem, G.-L. Zhao, L. Eriksson, A. Córdova, Chem. Eur. J., 2007, 13, 574; (o) Y. Hayashi, T. Okano, S. Aratake, D. Hazelard, Angew. Chem., Int. Ed., 2007, 46, 4922; (p) D. Enders, A. A. Narine, T. Benninghaus, G. Raabe, Synlett, 2007, 1667; (q) M. M. Biddle, M. Lin, K. A. Scheidt, J. Am. Chem., Soc. 2007, 129, 3830; (r) A. Chan, K. A. Scheidt, J. Am. Chem. Soc., 2007, 129, 5334; (s) M. Terada, K. Machioka, K. Sorimachi, J. Am. Chem. Soc., 2007, 129, 10336; (t) R. Dodda, J. J. Goldman, T. Mandal, C.-G. Zhao, G. A. Broker, E. R. T. Tiekink, Adv. Synth. Catal., 2008, 350, 537; (u) R. Dodda, Mandal, T. Mandal, C.-G. Zhao, Tetraheron Lett., 2008, 49, 1899; (v) Y.-H. Zhao, C.-W. Zheng, G. Zhao, W.-G. Cao, Tetrahedron: Asymmetry, 2008, 19, 701; (w) R. M. de Figueiredo, R. Froelich, M. Christmann, Angew. Chem., Int. Ed., 2008, 47, 1450; (x) N. T. Vo, R. D. Pace, M. F. O'Hara, M. J. Gaunt, J. Am. Chem. Soc., 2008, 130, 404.

10 Only a handful of examples of organocatalytic cascade Michael-Michael reactions: (a) Y. Hoashi, T. Yabuta, P. Yuan, H. Miyabe, Y. Takemoto, Tetrahedron 2006, 62, 365; (b) V. Sriramurthy, G. A. Barcan, O, Kwon, J. Am. Chem. Soc., 2007, 129, 12928; (c) X. Sun, S. Sengupta, J. L. Petersen, H. Wang, J. P. Lewis, X.-D. Shi, Org. Lett., 2007, 9, 4495; (d) H. Li, L. Zu, H. Xie, J. Wang, W. Jiang, W. Wang, Angew. Chem., Int. Ed., 2007, 46, 3732; (e) J. Wang, H. Xie, H. Li, L. Zu, W. Wang, Angew. Chem., Int. Ed., 2008, 47, 4177.
11 (a) Y. K. Chen, M. Yishida, D. W. C. MacMillan, J. Am. Chem. Soc., 2006, 128, 9328; (b) D. J. Guerin, S. J. Miller, J. Am Chem. Soc., 2002, 124, 2134.
12 For leading studies of chiral pyrrolinol ethers catalyzed reactions, see: (a) M. Marigo, T. C. Wabnitz, D. Fielenbach, K. A. Jørgensen, Angew. Chem. Int. Ed., 2005, 44, 794; (b) Y. Hayashi, H. Gotoh, T. Hayashi, M. Shoji, Angew. Chem., Int. Ed., 2005, 44, 4212; (c) Y. Chi, S. H. Gellman, Org. Lett., 2005, 7, 4253; (d) Y. Chi, S. H. Gellman, J. Am. Chem. Soc., 2006, 118, 6804; (e) H. Gotoh, R. Masui, H. Ogino, M. Shoji, Hayashi, Y Angew. Chem., Int. Ed., 2006, 45, 6853; (f) Y. Chi, E. P. English, W. C. Pomerantz, W. S. Horne, L. A. Joyce, L. R. Alexander, W. S. Fleming, E. A. Hopkins, S. H. Gellman, J. Am. Chem. Soc., 2007, 127, 6050.
13 CCDC-693305 contains the supplementary crystallographic data for this experiment. These data can be obtained free of charge via www.ccdc.cam.ac.uk and supporting informiation (reference article Li, et al., “Highly enantio- and diastereoselective organocatalytic cascade aza-Michael-Michael reactions: a direct method for the synthesis of trisubstituted chiral pyrrolidines”, Chem. Commun., 2008, 5636-5638, The Royal Society of Chemistry 2008).

1. A processes for synthesizing a compound of the formula 3(a) or formula 3(b): 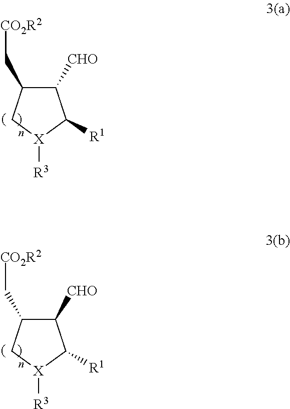
by reacting an α, β-unsaturated aldehyde of formula 1: 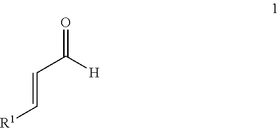
with a trans-γ-protected amino α, β-unsaturated ester of the formula 2 (a) or a trans-γ-mercapto α, β-unsaturated ester of the formula 2 (b): 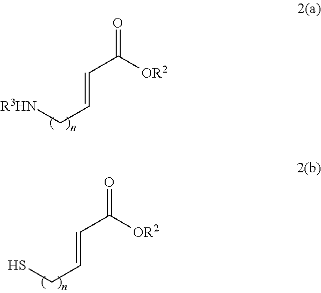
in a reaction medium comprising a weak base, a solvent, and a chiral ether catalyst of the formula 4(a) (to yield compounds of the formula 3(a)) or formula 4(b) (to yield compounds of the formula 3(b)): 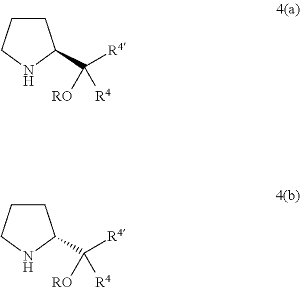
wherein:
n is 1 or 2;
X is N or S;
R¹ and R² are the same or different and are selected independently from the group consisting of an optionally substituted C₁-C₂₀ alkyl, alkenyl or alkynyl group, an alkoxy group, an optionally substituted aryl group, or an optionally substituted aralkyl group;
where (1) X is N, the α, β-unsaturated aldehyde of formula 1 is reacted with the trans-γ-protected amino α, β-unsaturated ester of the formula 2(a) and R³ is an amine protecting group, and (2) where X is S, the α, β-unsaturated aldehyde of formula 1 is reacted with the trans-γ-protected mercapto α, β-unsaturated ester of the formula 2(b) and R³ is absent;
R⁴ and R⁴′ are the same or different and are an optionally substituted aryl group or an optionally substituted aralkyl group; and
R is selected from the group consisting of an optionally substituted C₁-C₂₀ alkyl, alkenyl or alkynyl group and a compound of the formula 5: 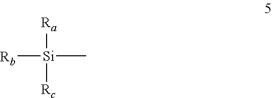
wherein Rₐ, R_{b}, and R_{c} are the same or different and are selected independently from the group consisting of an optionally substituted C₁-C₂₀ alkyl, alkenyl or alkynyl group, an alkoxy group, an optionally substituted aryl group, and an optionally substituted aralkyl group;
and wherein the concentration of the chiral ether catalyst of formula 4(a) or formula 4(b) in the reaction medium is between about 5 mole percent to about 30 mole percent and the process is done one-pot.

2. The process of claim 1, wherein R¹ is an optionally substituted aryl group and R² is an optionally substituted C₁-C₂₀ alkyl, alkenyl or alkynyl group.

3. The process of claim 1, wherein:
(a) R¹ is a phenyl group which is optionally substituted with up to four substituents selected from the group consisting of hydroxyl (OH), halogen, CN, NO₂, a C₁-C₆ optionally substituted alkyl group, a C₁-C₆ alkoxy group, and a C₂-C₆ ether group; and
(b) R² is an optionally substituted C₁-C₂₀ alkyl, alkenyl or alkynyl group.

4. The process of claim 1, wherein X is N and R³ is linear or cyclic and includes or constitutes one or more compounds selected from the group consisting of tosyl (p-toluenesulfonyl) (Ts), p-Methoxybenzyl carbonyl (Moz or MeOZ), 9-Fluorenylmethyloxycarbonyl (FMOC), benzyl (Bn), p-Methoxybenzyl (PMB) 3,4-dimethoxybenzyl (DMPM), p-methoxyphenyl (PMP), a sulfonamide tert-butyloxycarbonyl (BOC group), a carbobenzyloxy (CBZ group), a 2,4-nitrophenylsulfonyl group, a 2-nitrophenylsulfonyl group and a 4-nitophenyl sulfonyl group.

5. The process of claim 1, wherein:
(a) the solvent is selected from the group consisting of halogenated solvents, diethyl ether, water, pyridine, triethanolamine, tetrahydrofuran, 1,4-dioxane, acetonitrile, dimethylacetamide (DMA), dimethylformamide (DMF), dimethylsulfoxide (DMSO), acetonitrile, nitromethane, methanol, ethanol, isopropanol, methanol, ethanol, isopropanol, N-methylpyrrolidone (NMP), ethylacetate, benzene, toluene, tetrahydrofuran and mixtures thereof; and
(b) the weak bases is selected from the group consisting of phenolates and the ammonium, transition metal, alkali metal, and alkali earth metal salts of HCOO⁻, AcO⁻, CF₃COO⁻, tBuCOO⁻, HCO₃⁻. HSO₄⁻, SO₄²⁻, HSO₃⁻, H₂₂PO₃⁻, HPO₃²⁻.

6. The process of claim 5, wherein the halogenated solvents are selected from the group consisting of trichlorethylene, perchlorethylene (tetrachlorethylene), methylene chloride (CH₂Cl₂), carbon tetrachloride (CCl₄)), chloroform (CHCl₃), and 1,1,1-trichloroethane (methyl chloroform, CH₃—CCl).

7. The process of claim 1, wherein:
(a) R⁴ and R⁴′ are the same or different and are a phenyl group which is optionally substituted with up to four substituents selected from the group consisting of hydroxyl (OH), halogen, CN, NO₂, a C₁-C₆ optionally substituted alkyl group, a C₁-C₆ alkoxy group, and a C₂-C₆ ether group; and
(b) R is selected from the group consisting of trimethylsilyl (TMS), tert-butyldimethylsilyl (TBS), and triethylsilyl (TES).

8. The process of claim 1, wherein X is N and the process synthesizes a compound of the formula 3(a) by reacting an α, β-unsaturated aldehyde of formula 1 with a trans-γ-protected amino α, β-unsaturated ester of the formula 2(a) in a reaction medium comprising a weak base, a solvent, and a chiral ether catalyst of the formula 4(a), and wherein:
(a) the concentration of the chiral ether catalyst of formula 4(a) in the reaction medium is between about 10 mole percent to about 20 mole percent;
(b) the enantioselectivity of the process is between about 96% to greater than about 99%.

9. The process of claim 8, wherein the concentration in the reaction medium of the chiral ether catalyst of formula 4(a) is about 20 mole %.

10. The process of claim 1, wherein X is N and the process further comprises the step of deprotecting the compound of formula 3(a) or formula 3(b) by removing the R³ moiety.

11. A processes for synthesizing a compound of the formula 3(a) or formula 3(b): 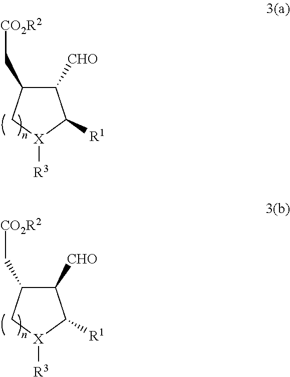
by reacting an α, β-unsaturated aldehyde of formula 1: 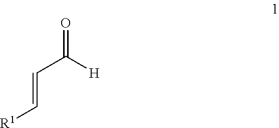
with a trans-γ-protected amino α, β-unsaturated ester of the formula 2 (a) or a trans-γ-mercapto α, β-unsaturated ester of the formula 2 (b): 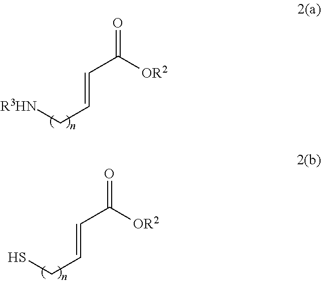
in a reaction medium comprising a weak base, a solvent, and a chiral ether catalyst of the formula 4(a) (to yield a compound of the formula 3(a)) or formula 4(b) (to yield a compound of the formula 3(b)): 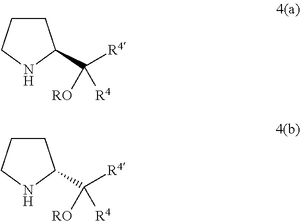
wherein:
n is 1 or 2;
X is N or S;
R¹ and R² are the same or different and are selected independently from the group consisting of an optionally substituted C₁-C₂₀ alkyl, alkenyl or alkynyl group, an alkoxy group, an optionally substituted aryl group, or an optionally substituted aralkyl group;
where (1) X is N, the α, β-unsaturated aldehyde of formula 1 is reacted with the trans-γ-protected amino α, β-unsaturated ester of the formula 2(a) and R³ is an amine protecting group, and (2) where X is S, the α, β-unsaturated aldehyde of formula 1 is reacted with the trans-γ-protected mercapto α, β-unsaturated ester of the formula 2(b) and R³ is absent;
R⁴ and R⁴′ are the same or different and are an optionally substituted aryl group or an optionally substituted aralkyl group; and
R is selected from the group consisting of an optionally substituted C₁-C₂₀ alkyl, alkenyl or alkynyl group, trimethylsilyl (TMS), tert-butyldimethylsilyl (TBS), t-butyldimethylsilyl (TBDMS), t-butyldiphenylsilyl (TBDPS), triethylsilyl (TES), tert-butyldimethylsilyloxymethyl (TOM), triisopropylsilyl (TIPS), bis(trimethylsilyl)acetamide (BSA), triphenylsilyl, tetraisopropyldisiloxan-1,3-diyl, pivaloyl, acetyl (Ac), benzoyl (Bz), benzyl (Bn), β-methoxyethoxymethyl ether (MEM), trityl (triphenylmethyl), methoxytrityl, dimethoxytrityl (bis-(4 methoxyphenyl)phenylmethyl) (DMT), methoxytrityl (4-methoxyphenyl diphenylmethyl (MMT)), methoxymethyl ether (MOM), p-methoxybenzyl ether (PMB), methylthiomethyl ether, tetrahydropyranyl (THP), a methyl ether, and an ethoxyethyl ether;
and wherein the concentration of the chiral ether catalyst of formula 4(a) or formula 4(b) in the reaction medium is between about 5 mole percent to about 30 mole percent and the process is done one-pot.

12. The process of claim 11, wherein R¹ is an optionally substituted aryl group and R² is an optionally substituted C₁-C₂₀ alkyl, alkenyl or alkynyl group.

13. The process of claim 11, wherein:
(a) R¹ is a phenyl group which is optionally substituted with up to four substituents selected from the group consisting of hydroxyl (OH), halogen, CN, NO₂, a C₁-C₆ optionally substituted alkyl group, a C₁-C₆ alkoxy group, and a C₂-C₆ ether group; and
(b) R² is an optionally substituted C₁-C₂₀ alkyl, alkenyl or alkynyl group.

14. The process of claim 11, wherein X is N and R³ is linear or cyclic and includes or constitutes one or more compounds selected from the group consisting of tosyl (p-toluenesulfonyl) (Ts), p-Methoxybenzyl carbonyl (Moz or MeOZ), 9-Fluorenylmethyloxycarbonyl (FMOC), benzyl (Bn), p-Methoxybenzyl (PMB) 3,4-dimethoxybenzyl (DMPM), p-methoxyphenyl (PMP), a sulfonamide, tert-butyloxycarbonyl (BOC group), a carbobenzyloxy (CBZ group), a 2,4-nitrophenylsulfonyl group, a 2-nitrophenylsulfonyl group and a 4-nitophenyl sulfonyl group.

15. The process of claim 11, wherein:
(a) the solvent is selected from the group consisting of halogenated solvents, diethyl ether, water, pyridine, triethanolamine, tetrahydrofuran, 1,4-dioxane, acetonitrile, dimethylacetamide (DMA), dimethylformamide (DMF), dimethylsulfoxide (DMSO), acetonitrile, nitromethane, methanol, ethanol, isopropanol, methanol, ethanol, isopropanol, N-methylpyrrolidone (NMP), ethylacetate, benzene, toluene, tetrahydrofuran and mixtures thereof; and
(b) the weak bases is selected from the group consisting of phenolates and the ammonium, transition metal, alkali metal, and alkali earth metal salts of HCOO⁻, AcO⁻, CF₃COO⁻, tBuCOO⁻, HCO₃⁻. HSO₄⁻, SO₄²⁻, HSO₃⁻, H₂₂PO₃⁻, HPO₃²⁻.

16. The process of claim 15, wherein the halogenated solvents are selected from the group consisting of trichlorethylene, perchlorethylene (tetrachlorethylene), methylene chloride (CH₂Cl₂), carbon tetrachloride (CCl₄)), chloroform (CHCl₃), and 1,1,1-trichloroethane (methyl chloroform, CH₃—CCl).

17. The process of claim 11, wherein:
(a) R⁴ and R⁴′ are the same or different and are a phenyl group which is optionally substituted with up to four substituents selected from the group consisting of hydroxyl (OH), halogen, CN, NO₂, a C₁-C₆ optionally substituted alkyl group, a C₁-C₆ alkoxy group, and a C₂-C₆ ether group; and
(b) R is selected from the group consisting of trimethylsilyl (TMS), tert-butyldimethylsilyl (TBS), and triethylsilyl (TES).

18. The process of claim 11, wherein X is N and the process synthesizes a compound of the formula 3(a) by reacting an α, β-unsaturated aldehyde of formula 1 with a trans-γ-protected amino α, β-unsaturated ester of the formula 2 in a reaction medium comprising a weak base, a solvent, and a chiral ether catalyst of the formula 4(a), and wherein:
(a) the concentration of chiral ether catalyst of formula 4(a) in the reaction medium is between about 10 mole percent to about 20 mole percent;
(b) the enantioselectivity of the process is between about 96% to greater than about 99%.

19. The process of claim 18, wherein the concentration in the reaction medium of the chiral ether catalyst of formula 4(a) is about 20 mole %.

20. The process of claim 11, wherein X is N and the process further comprises the step of deprotecting the compound of formula 3(a) or formula 3(b) by removing the R³ moiety.

21. A processes for synthesizing a compound of the formula 6: 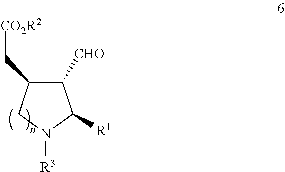
by reacting an α, β-unsaturated aldehyde of formula 1: 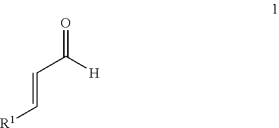
with a trans-γ-protected amino α, β-unsaturated ester of the formula 2(a): 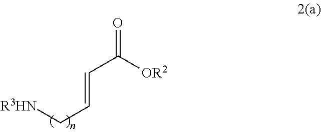
in a reaction medium comprising a weak base, a solvent, and a chiral ether catalyst of the formula 4(a): 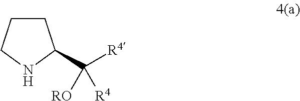
wherein:
n is 1 or 2;
R¹ and R² are the same or different and are selected independently from the group consisting of an optionally substituted C₁-C₂₀ alkyl, alkenyl or alkynyl group, an alkoxy group, an optionally substituted aryl group, or an optionally substituted aralkyl group;
R³ is an amine protecting group;
R⁴ and R⁴′ are the same or different and are an optionally substituted aryl group or an optionally substituted aralkyl group; and
R is selected from the group consisting of an optionally substituted C₁-C₂₀ alkyl, alkenyl or alkynyl group, trimethylsilyl (TMS), tert-butyldimethylsilyl (TBS), t-butyldimethylsilyl (TBDMS), t-butyldiphenylsilyl (TBDPS), triethylsilyl (TES), tert-butyldimethylsilyloxymethyl (TOM), triisopropylsilyl (TIPS), bis(trimethylsilyl)acetamide (BSA), triphenylsilyl, tetraisopropyldisiloxan-1,3-diyl, pivaloyl, acetyl (Ac), benzoyl (Bz), benzyl (Bn), β-methoxyethoxymethyl ether (MEM), trityl (triphenylmethyl), methoxytrityl, dimethoxytrityl (bis-(4 methoxyphenyl)phenylmethyl) (DMT), methoxytrityl (4-methoxyphenyl diphenylmethyl (MMT)), methoxymethyl ether (MOM), p-methoxybenzyl ether (PMB), methylthiomethyl ether, tetrahydropyranyl (THP), a methyl ether, and an ethoxyethyl ether; and
wherein the concentration of the chiral ether catalyst of formula 7 in the reaction medium is between about 5 mole percent to about 30 mole percent and the process is done one-pot.

22. The process of claim 21, wherein R¹ is an optionally substituted aryl group and R² is an optionally substituted C₁-C₂₀ alkyl, alkenyl or alkynyl group.

23. The process of claim 21, wherein:
(a) R¹ is a phenyl group which is optionally substituted with up to four substituents selected from the group consisting of hydroxyl (OH), halogen, CN, NO₂, a C₁-C₆ optionally substituted alkyl group, a C₁-C₆ alkoxy group, and a C₂-C₆ ether group; and
(b) R² is an optionally substituted C₁-C₂₀ alkyl, alkenyl or alkynyl group.

24. The process of claim 21, wherein R³ is linear or cyclic and includes or constitutes one or more compounds selected from the group consisting of tosyl (p-toluenesulfonyl) (Ts), p-Methoxybenzyl carbonyl (Moz or MeOZ), 9-Fluorenylmethyloxycarbonyl (FMOC), benzyl (Bn), p-Methoxybenzyl (PMB) 3,4-dimethoxybenzyl (DMPM), p-methoxyphenyl (PMP), a sulfonamide, tert-butyloxycarbonyl (BOC group), a carbobenzyloxy (CBZ group), a 2,4-nitrophenylsulfonyl group, a 2-nitrophenylsulfonyl group and a 4-nitophenyl sulfonyl group.

25. The process of claim 21, wherein:
(a) the solvent is selected from the group consisting of halogenated solvents, diethyl ether, water, pyridine, triethanolamine, tetrahydrofuran, 1,4-dioxane, acetonitrile, dimethylacetamide (DMA), dimethylformamide (DMF), dimethylsulfoxide (DMSO), acetonitrile, nitromethane, methanol, ethanol, isopropanol, methanol, ethanol, isopropanol, N-methylpyrrolidone (NMP), ethylacetate, benzene, toluene, tetrahydrofuran and mixtures thereof; and
(b) the weak base is selected from the group consisting of phenolates and the ammonium, transition metal, alkali metal, and alkali earth metal salts of HCOO⁻, AcO⁻, CF₃COO⁻, tBuCOO⁻, HCO₃⁻. HSO₄⁻, SO₄²⁻, H₂2PO₃⁻, HPO₃²⁻.

26. The process of claim 25, wherein the halogenated solvents are selected from the group consisting of trichlorethylene, perchlorethylene (tetrachlorethylene), methylene chloride (CH₂Cl₂), carbon tetrachloride (CCl₄)), chloroform (CHCl₃), and 1,1,1-trichloroethane (methyl chloroform, CH₃—CCl).

27. The process of claim 21, wherein:
(a) R⁴ and R⁴′ are the same or different and are a phenyl group which is optionally substituted with up to four substituents selected from the group consisting of hydroxyl (OH), halogen, CN, NO₂, a C₁-C₆ optionally substituted alkyl group, a C₁-C₆ alkoxy group, and a C₂-C₆ ether group; and
(b) R is selected from the group consisting of trimethylsilyl (TMS), tert-butyldimethylsilyl (TBS), and triethylsilyl (TES).

28. The process of claim 21, wherein the process synthesizes a compound of the formula 6 by reacting an α, β-unsaturated aldehyde of formula 1 with a trans-γ-protected amino α, β-unsaturated ester of the formula 2(a) in a reaction medium comprising a weak base, a solvent, and a chiral ether catalyst of the formula 4(a), and wherein:
(a) R¹ is a phenyl group which is optionally substituted with up to four substituents which are selected from the group consisting of hydroxyl (OH), halogen, CN, NO₂, a C₁-C₆ optionally substituted alkyl group, a C₁-C₆ alkoxy group, or a C₂-C₆ ether group;
(b) R² is an optionally substituted C₁-C₂₀ alkyl, alkenyl or alkynyl group;
(c) R³ is tosyl (p-toluenesulfonyl) (Ts);
(d) R⁴ and R⁴′ are the same or different and are a phenyl group which is optionally substituted with up to four substituents which are selected from the group consisting of hydroxyl (OH), halogen, CN, NO₂, a C₁-C₆ optionally substituted alkyl group, a C₁-C₆ alkoxy group, and a C₂-C₆ ether group;
(e) R is selected from the group consisting of trimethylsilyl (TMS), tert-butyldimethylsilyl (TBS), and triethylsilyl (TES);
(f) the concentration of the chiral ether catalyst of formula 7 in the reaction medium is between about 10 mole percent to about 20 mole percent;
(g) the enantioselectivity of the process is between about 96% to greater than about 99%.

29. The process of claim 21, wherein the process synthesizes a compound of the formula 6 by reacting an α, β-unsaturated aldehyde of formula 1 with a trans-γ-protected amino α, β-unsaturated ester of the formula 2(a) in a reaction medium comprising a weak base, a solvent, and a chiral ether catalyst of the formula 4(a), and wherein:
(a) R¹ is a phenyl group which is optionally substituted with up to four substituents which are selected from the group consisting of hydroxyl (OH), halogen, CN, NO₂, a C₁-C₆ optionally substituted alkyl group, a C₁-C₆ alkoxy group, or a C₂-C₆ ether group;
(b) R² is an optionally substituted C₁-C₂₀ alkyl, alkenyl or alkynyl group;
(c) R³ is tosyl (p-toluenesulfonyl) (Ts);
(d) R⁴ and R⁴′ are a phenyl group;
(e) R is selected from the group consisting of trimethylsilyl (TMS), tert-butyldimethylsilyl (TBS), and triethylsilyl (TES);
(g) the solvent is methylene chloride (CH₂Cl₂);
(g) the weak base is sodium acetate (NaOAc);
(h) the concentration in the reaction medium of the chiral diarylprolinol ether of formula 7 is about 20 mole %;
(i) the enantioselectivity of the process is between about 96% to greater than about 99%.

30. The process of claim 21, wherein the process further comprises the step of deprotecting the compound of formula 6 by removing the R³ moiety.

31. The process of claim 21, wherein n is 1.

32. A processes for synthesizing a compound of the formula 8: 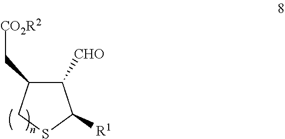
by reacting an α, β-unsaturated aldehyde of formula 1: 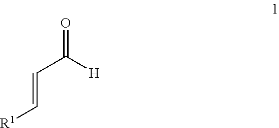
with trans-γ-mercapto α, β-unsaturated ester of the formula 2 (b): 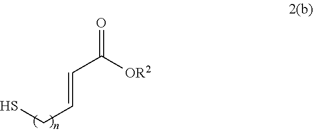
in a reaction medium comprising a weak base, a solvent, and a chiral ether catalyst of the formula 4(a): 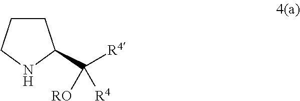
wherein:
n is 1 or 2;
R′ and R² are the same or different and are selected independently from the group consisting of an optionally substituted C₁-C₂₀ alkyl, alkenyl or alkynyl group, an alkoxy group, an optionally substituted aryl group, or an optionally substituted aralkyl group;
R⁴ and R⁴′ are the same or different and are an optionally substituted aryl group or an optionally substituted aralkyl group; and
R is selected from the group consisting of an optionally substituted C₁-C₂₀ alkyl, alkenyl or alkynyl group, trimethylsilyl (TMS), tert-butyldimethylsilyl (TBS), t-butyldimethylsilyl (TBDMS), t-butyldiphenylsilyl (TBDPS), triethylsilyl (TES), tert-butyldimethylsilyloxymethyl (TOM), triisopropylsilyl (TIPS), bis(trimethylsilyl)acetamide (BSA), triphenylsilyl, tetraisopropyldisiloxan-1,3-diyl, pivaloyl, acetyl (Ac), benzoyl (Bz), benzyl (Bn), β-methoxyethoxymethyl ether (MEM), trityl (triphenylmethyl), methoxytrityl, dimethoxytrityl (bis-(4 methoxyphenyl)phenylmethyl) (DMT), methoxytrityl (4-methoxyphenyl diphenylmethyl (MMT)), methoxymethyl ether (MOM), p-methoxybenzyl ether (PMB), methylthiomethyl ether, tetrahydropyranyl (THP), a methyl ether, and an ethoxyethyl ether;
and wherein the concentration of the chiral ether catalyst of formula 9 in the reaction medium is between about 5 mole percent to about 30 mole percent and the process is done one-pot.

33. The process of claim 32, wherein R¹ is an optionally substituted aryl group and R² is an optionally substituted C₁-C₂₀ alkyl, alkenyl or alkynyl group.

34. The process of claim 32, wherein:
(a) R¹ is a phenyl group which is optionally substituted with up to four substituents selected from the group consisting of hydroxyl (OH), halogen, CN, NO₂, a C₁-C₆ optionally substituted alkyl group, a C₁-C₆ alkoxy group, and a C₂-C₆ ether group; and
(b) R² is an optionally substituted C₁-C₂₀ alkyl, alkenyl or alkynyl group.

35. The process of claim 32, wherein R³ is linear or cyclic and includes or constitutes one or more compounds selected from the group consisting of acetyl (Ac), benzoyl, methoxyacetyl, 1-3-dioxacyclohexyl, 1,3-dioxacyclopentyl, alkoxycarbonyl, carbamoyl, alkoxyalkyl, dialkoxyalkyl, tetrahydropyranyl, tetrahydrofuranyl, p-methoxybenzyl, benzhydryl, trityl, and (trimethylsilyl)ethoxymethyl (SEM).

36. The process of claim 32, wherein:
(a) the solvent is selected from the group consisting of halogenated solvents, diethyl ether, water, pyridine, triethanolamine, tetrahydrofuran, 1,4-dioxane, acetonitrile, dimethylacetamide (DMA), dimethylformamide (DMF), dimethylsulfoxide (DMSO), acetonitrile, nitromethane, methanol, ethanol, isopropanol, methanol, ethanol, isopropanol, N-methylpyrrolidone (NMP), ethylacetate, benzene, toluene, tetrahydrofuran and mixtures thereof; and
(b) the weak bases is selected from the group consisting of phenolates and the ammonium, transition metal, alkali metal, and alkali earth metal salts of HCOO⁻, AcO⁻, CF₃COO⁻, tBuCOO⁻, HCO₃⁻. HSO₄⁻, SO₄²⁻, HSO₃⁻, H₂2PO₃⁻, HPO₃²⁻.

37. The process of claim 36, wherein the halogenated solvents are selected from the group consisting of trichlorethylene, perchlorethylene (tetrachlorethylene), methylene chloride (CH₂Cl₂), carbon tetrachloride (CCl₄)), chloroform (CHCl₃), and 1,1,1-trichloroethane (methyl chloroform, CH₃—CCl).

38. The process of claim 32, wherein:
(a) R⁴ and R⁴′ are the same or different and are a phenyl group which is optionally substituted with up to four substituents selected from the group consisting of hydroxyl (OH), halogen, CN, NO₂, a C₁-C₆ optionally substituted alkyl group, a C₁-C₆ alkoxy group, and a C₂-C₆ ether group; and
(b) R is selected from the group consisting of trimethylsilyl (TMS), tert-butyldimethylsilyl (TBS), and triethylsilyl (TES).

39. The process of claim 32, wherein the process synthesizes a compound of the formula 8 by reacting an α, β-unsaturated aldehyde of formula 1 with a trans-γ-protected mercapto α, β-unsaturated ester of formula 2(b) in a reaction medium comprising a weak base, a solvent, and a chiral ether catalyst of the formula 4(a), and wherein:
(a) R¹ is a phenyl group which is optionally substituted with up to four substituents which are selected from the group consisting of hydroxyl (OH), halogen, CN, NO₂, a C₁-C₆ optionally substituted alkyl group, a C₁-C₆ alkoxy group, or a C₂-C₆ ether group;
(b) R² is an optionally substituted C₁-C₂₀ alkyl, alkenyl or alkynyl group;
(c) R⁴ and R⁴′ are the same or different and are a phenyl group which is optionally substituted with up to four substituents which are selected from the group consisting of hydroxyl (OH), halogen, CN, NO₂, a C₁-C₆ optionally substituted alkyl group, a C₁-C₆ alkoxy group, and a C₂-C₆ ether group;
(d) R is selected from the group consisting of trimethylsilyl (TMS), tert-butyldimethylsilyl (TBS), and triethylsilyl (TES);
(e) the concentration of the chiral ether catalyst of formula 9 in the reaction medium is between about 10 mole percent to about 20 mole percent;
(f) the enantioselectivity of the process is between about 96% to greater than about 99%.

40. The process of claim 32, wherein the process synthesizes a compound of the formula 8 by reacting an α, β-unsaturated aldehyde of formula 1 with a trans-γ-protected mercapto α, β-unsaturated ester of formula 2(b) in a reaction medium comprising a weak base, a solvent, and a chiral ether catalyst of the formula 4(a), and wherein:
(a) R¹ is a phenyl group which is optionally substituted with up to four substituents which are selected from the group consisting of hydroxyl (OH), halogen, CN, NO₂, a C₁-C₆ optionally substituted alkyl group, a C₁-C₆ alkoxy group, or a C₂-C₆ ether group;
(b) R² is an optionally substituted C₁-C₂₀ alkyl, alkenyl or alkynyl group;
(c) R⁴ and R⁴′ are a phenyl group;
(d) R is selected from the group consisting of trimethylsilyl (TMS), tert-butyldimethylsilyl (TBS), and triethylsilyl (TES);
(e) the solvent is methylene chloride (CH₂Cl₂);
(f) the weak base is sodium acetate (NaOAc);
(g) the concentration in the reaction medium of the chiral ether catalyst of formula 9 is about 20 mole %;
(h) the enantioselectivity of the process is between about 96% to greater than about 99%.

41. The process of claim 32, wherein R is TMS.

42. The process of claim 32, wherein n is 1.

43. A catalyst of the formula 4(a) or formula 4(b): 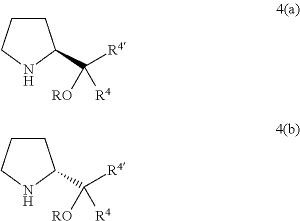
wherein:
R⁴ and R⁴′ are the same or different and are an optionally substituted aryl group or an optionally substituted aralkyl group; and
R is selected from the group consisting of an optionally substituted C₁-C₂₀ alkyl, alkenyl or alkynyl group and a compound of the formula 5 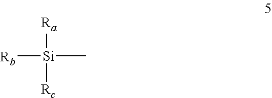
wherein Rₐ, R_{b}, and R_{c} are the same or different and are selected independently from the group consisting of an optionally substituted C₁-C₂₀ alkyl, alkenyl or alkynyl group, an alkoxy group, an optionally substituted aryl group, and an optionally substituted aralkyl group.

44. (canceled)

45. (canceled)

46. (canceled)

47. (canceled)

48. (canceled)

49. (canceled)

50. (canceled)
